(19) United States
(12) Patent Application Publication
(10) Pub. No.: US 2022/0041997 A1
(43) Pub. Date: Feb. 10, 2022

(54) Method for rescuing influenza virus and composition therefor

(71) Applicant: Zhejiang Senwei Biomedical Development Co., Ltd, Xianning (CN)

(72) Inventors: Dongsheng Dai, Xianning (CN); Xun Li, Xianning (CN); Xin Tian, Xianning (CN)

(21) Appl. No.: 17/471,709

(22) Filed: Sep. 10, 2021

Related U.S. Application Data
(63) Continuation of application No. PCT/CN2019/121905, filed on Nov. 29, 2019.

(30) Foreign Application Priority Data
Mar. 13, 2019 (CN) 201910188724

(51) Int. Cl. C12N 7/00; C12N 9/12; C12N 9/24; C12N 15/85; C12N 5/071; A61K 39/145; C07K 14/005

(52) U.S. Cl. CPC C12N 7/00; C12N 9/1241; C12Y 207/07038; C12N 9/2402; C12Y 302/01018; C12N 15/85; C12N 2760/00052; A61K 39/145; C07K 14/005; C12N 2760/16121; C12N 2760/16134; C12N 2760/00043; C12N 5/0686

(57) ABSTRACT

The present invention relates to a new method for rescuing an influenza virus and a composition therefor. The method comprises providing a host cell stably integrated with and expressing influenza virus PA, PB1, PB2 and NP genes, and introducing an influenza virus rescue system in which a stop codon is introduced into the PA, PB1, PB2 and NP genes respectively into the host cell to achieve virus rescue. The produced virus particles can be used as a live attenuated influenza vaccine, which is characterized in that, since the genes encoding the related proteins are mutated, it has no replication and proliferation ability in human and normal animal cells, and replication and proliferation can be achieved only in the host cells constructed above and it can fully stimulate the body immunity and effectively protect the body while ensuring the safety.

### TECHNICAL FIELD

The present invention relates to the field of biotechnology, and in particular to a method for rescuing a replication-controllable influenza virus and a composition used in the method.

### BACKGROUND ART

Influenza (flu) is a disease of the respiratory tract and other organs caused by influenza virus. There are varying degrees of prevalence in every spring and winter, and even in other seasons among healthy children and adults. It is usually an acute, infectious disease.

Influenza virus is the pathogen causing the flu. It belongs to the negative-sense single-stranded RNA virus. Its genome consists of 8 independent RNA fragments (named as fragments 1-8 respectively), and the total length of the nucleic acid is about 13.6 kb. These 8 fragments encode a total of 10 proteins, 8 of which are structural proteins, including PB1, PB2, PA, HA, NA, NP, M1 and M2, while NS1 and NS2 are non-structural proteins. Influenza viruses are classified into human influenza virus and animal influenza virus, and human influenza viruses are classified into three types: A, B, and C.

Viral replication relies primarily on viral ribonucleoproteins (vRNPs). The ribonucleoprotein of influenza A virus is composed of viral RNA, RNA polymerase (RdRp) complex and nuclear protein (NP), which is the smallest unit of replication of the virus, and viral proteins can only be expressed on the basis of this structure. The RdRp in the vRNPs structure consists of three subunits (PA, PB2 and PB1). PB1 is located at the core of the trimer, and it forms a stable protein complex by forming non-covalent bonds (such as hydrophobic interaction, hydrogen bonding, van der Waals force, etc.) with the C-terminus of the PA subunit and the N-terminus of the PB2 subunit through its N-terminus and C-terminus, respectively.

Although various types of antiviral drugs can be used to treat influenza viruses, due to the rapid mutation of influenza viruses, influenza epidemics and outbreaks occur every year around the world. Correct vaccination can effectively reduce the incidence rate of influenza.

Currently, influenza vaccines can be classified into three categories according to their types: whole inactivated virus vaccines, split vaccines, and subunit vaccines. Influenza whole inactivated virus vaccine has high immunogenicity and relatively low production cost, but the incidence rate of side effects during vaccination is also high, and should not be applied to children under 6 years of age, these all limit the application of influenza whole virus vaccine. The split vaccine is based on the influenza whole inactivated virus vaccine, which is prepared by selecting the appropriate split agent and split conditions to split the influenza virus, purifying to remove viral nucleic acids and macromolecular proteins and retaining antigen active components HA and NA and part of M protein and NP protein. The split influenza vaccine can reduce the vaccination side effect of the whole inactivated virus vaccine and maintain relatively high immunogenicity, which can expand the scope of use of the vaccine. But the split agent must be added and removed during the preparation process, moreover a large amount of antigen is lost during the split process, resulting in a decrease in the protection efficiency of influenza vaccine. Based on the split vaccine, virion subunit and surface antigen (HA and NA) vaccines have been developed. The subunit influenza vaccine has a very pure antigenic component, but the influenza vaccine mutates quickly, therefore the preventive effect has been affected to some extent.

In addition, for whole inactivated virus vaccine and split vaccine, because influenza mutates rapidly, it is necessary to predict the strain that possibly breaks out for the year before the outbreak season every year. Accurate prediction has certain difficulties, and prediction error will lead to a significant decline in vaccine protection rate. Moreover, even if accurate prediction is made, also because of currently primarily used chicken embryo production process, the chicken embryo-adapted variation in viruses leads to inefficient vaccine protection efficiency; for subunit vaccines, progress has been slow due to the fact that it has not been discovered to date that influenza viruses have conserved sequences that can be used for vaccines.

In terms of production methods, the traditional method for preparing influenza virus vaccines is to use chicken embryo preparation. However, influenza vaccines prepared using chicken embryos still have production technical and safety problems. For example, the culture cycle is too long, the virus has variability during the cultivation process, the labor intensity is high, the efficiency is low, the production cost is high, and it is not easy to control the yield, and the difference between different batches of chicken embryos is large which is unfavorable to expand production to cope with large-scale flu outbreaks; on the other hand, there are quality and safety hazards in the vaccines produced by the chicken embryos themselves contaminated with bacteria or other viruses, and the number of waste embryos is large after the vaccine is produced, and the harmless treatment is difficult, involving biosafety and public health problems.

Therefore, there is an urgent need for an influenza vaccine which is safer and can better preserve the whole virus immune activity and its production method.

### SUMMARY OF THE INVENTION

The object of the present invention is to produce influenza virus by specific mammalian cells for vaccine preparation. The influenza virus cannot proliferate in normal mammalian cells due to introduction of mutations, and only can proliferate in host cells in which foreign viral protein genes are integrated.

In order to achieve the above object, the present invention transfers specific genes into a mammalian cell to obtain a host cell stably expressing the corresponding influenza virus proteins, and then transfects with the influenza virus rescue system in which related genes are mutated, and rescues a new type of replication-controllable live influenza virus. The preparation of the above live virus can provide a basis for the production of a safe live virus vaccine which does not replicate and proliferate in normal human and animal somatic cells.

Specifically, the present invention provides the following technical solution: a method for rescuing an influenza virus, comprising providing a mammalian host cell stably expressing influenza virus PA, PB1, PB2 and NP genes, introducing an influenza virus rescue system comprising mutant PA, PB1, PB2 and NP genes into the aforementioned host cell to achieve rescue, wherein the mutations make the influenza virus rescue system unable to rescue intact virus in natural mammalian cells.

Further, the method of the present invention can comprise the following steps:
(1) constructing a single- or multiple-plasmid system encoding the PA, PB1, PB2 and NP genes;
(2) introducing the single- or multiple-plasmid system of step (1) into a mammalian cell, and screening a host cell stably expressing the four genes, preferably, introducing by electrotransformation;
(3) constructing recombinant plasmids containing the mutant PA, PB1, PB2 and NP genes and recombinant plasmids encoding the four genes HA, NA, M and NS respectively to form an influenza virus rescue system, the mutations are achieved by introducing a TAG codon into each of the four gene sequences;
(4) co-transfecting the virus rescue system constructed in step (3) into the host cell of step (2);
(5) culturing the cell obtained in step (4) and harvesting the particles of the influenza virus.

Preferably, the foreign genes stably integrated into the host cell and the virus-encoding genes in the influenza virus rescue system of the present invention are all derived from the A/WSN/1933 strain of influenza virus H1N1.

Preferably, the nucleotide sequences of the PA, PB1, PB2 and NP genes in step (1) are as shown in SEQ ID NO: 1, SEQ ID NO: 2, SEQ ID NO: 3 and SEQ ID NO: 4, respectively.

Preferably, the influenza virus rescue system comprises the following eight plasmids: pPolI-M-PB2, pPolI-M-PB1, pPolI-M-PA, pPolI-M-NP, pPolI-WSN-HA, pPolI-WSN-NA, pPolI-WSN-M and pPolI-WSN-NS, wherein, the PA gene contained has a mutation at R266 codon to TAG, the PB1 gene contained has a mutation at R52 codon to TAG, the PB2 gene contained has a mutation at K33 codon to TAG, and the NP gene contained has a mutation at D101 codon to TAG.

The mammalian cells used in the present invention are preferably Vero cells, MDCK cells, 293 cells or MRCS cells, more preferably Vero cells or MDCK cells.

Further, the present invention also provides a host cell prepared according to step (2) of the above method.

The present invention also provides an influenza virus prepared according to the above method.

The present invention further provides an immunogenic composition comprising the influenza virus described above.

The present invention also provides a virus rescue composition comprising a host cell and a virus rescue system, wherein the host cell expresses PA, PB1, PB2 and NP genes, and the viral rescue system comprises mutant PA, PB1, PB2 and NP genes, the mutations make the influenza virus rescue system unable to rescue intact virus in natural mammalian cells

As a use of the technical solution of the present invention, also provided is the use of the above host cell, influenza virus or immunogenic composition, virus rescue composition for the preparation of a medicament for preventing or treating influenza. Preferably, the medicament is a vaccine.

The related terms are further explained below.

### Influenza Virus Strain

In the present invention, the gene fragments involved are derived from influenza A virus or influenza B virus, preferably from type A H1N1 (such as A/WSN/1933, A/PR/8), H3N2 (such as A/Aichi/2/68) virus strains.

As a preferred specific embodiment, the present invention uses an influenza virus strain of A/WSN/1933 from H1N1 as a source of cloning of all gene fragments. Preferably, the gene fragments are codon optimized. In a specific technical solution, the present invention performs codon optimization based on the preferences of Vero cells.

Many organisms show the preference for the use of specific codons, so genes can be regulated based on codon optimization for optimal gene expression in a given organism. The present invention finds that the gene sequence encoding the viral protein has codon bias between different host cells, and the preference directly affects the amount of protein expression and determines the efficiency of virus rescue. In a preferred embodiment, the inventors obtained optimized codons with higher levels of foreign protein expression and higher virus rescue efficiency through repeated trial and error, and based on codon optimization results: the PA gene sequence is as shown in SEQ ID NO: 1; the PB1 gene sequence is as shown in SEQ ID NO: 2; the PB2 gene sequence is as shown in SEQ ID NO: 3; and the NP gene sequence is as shown in SEQ ID NO: 4.

SEQ ID NO: 1 is as follows:

atggaggacttcgtgaggcagtgcttcaaccccatgatcgtggagctggc
cgagaaggccatgaaggagtacggcgaggacctgaagatcgagaccaaca
agttcgccgccatctgcacacacctggaggtgtgcttcatgtactctgac
ttccacttcatcgacgagcagggcgagtccatcgtggtggagctggggga
ccccaacgctctgctgaagcaccggttcgagatcatcgagggacgggaca
ggaccatcgcctggacagtgatcaacagcatctgcaacaccacaggggcc
gagaagcccaagttcctgcccgacctgtacgactacaagaagaacaggtt
catcgagatcggcgtgaccaggagagaggtgcacatctactacctggaga
aggccaacaagatcaagtccgagaagacccacatccacatcttcagcttc
acaggggaggagatggccaccaaggccgactacacactggacgaggagag
cagggcccggatcaagaccaggctgttcacaatcagacaggagatggcct
ccaggggcctgtgggactccttccggcagagcgagaggggagaggagaca
atcgaggagagattcgagatcaccggcacaatgagaaagctggccgacca
gtccctgccacccaacttcagctctctggagaagttcagagcctacgtgg
acgggttcgagcccaacggctacatcgaggggaagctgagccagatgtct
aaggaggtgaacgccagaatcgagcccttcctgaagagcacccccaggcc
cctgagactgccagacggaccaccatgctcccagcggagcaagttcctgc
tgatggacgccctgaagctgtccatcgaggaccccagccacgagggagag
ggcatccccctgtacgacgccatcaagtgcatgagaacattcttcggctg
gaaggagcccaacgtggtgaagccccacgagaaggggatcaaccccaact
acctgctgagctggaagcaggtgctggccgagctgcaggacatcgagaac
gaggagaagatcccccggaccaagaacatgaagaagacatctcagctgaa
gtgggctctgggagagaacatggctccagagaaggtggacttcgacgact
gcaaggacgtgggggacctgaagcagtacgacagcgacgagcccgagctg
aggtctctggcctcctggattcagaacgagttcaacaaggcctgcgagct
gaccgactccagctggatcgagctggacgagatcggagaggacgctgctc
ccatcgagcacatcgccagcatgcggaggaactacttcaccgccgaggtg
tctcactgcagggccacagagtacatcatgaagggcgtgtacatcaacac
agccctgctgaacgcctcctgcgctgctatggacgacttccagctgatcc
ccatgatcagcaagtgcagaaccaaggaggggagacggaagacaaacctg
tacggcttcatcatcaagggcaggagccacctgcggaacgacaccgacgt
ggtgaacttcgtgtctatggagttctccctgacagacccccggctggagc
cacacaagtgggagaagtactgcgtgctggaggtcggcgacatgctgctg
aggtctgccatcgggcacgtgtcccggcccatgttcctgtacgtgaggac
caacggcacaagcaagatcaagatgaagtgggggatggagatgaggagat
gcctgctgcagagcctgcagcagatcgagtctatgatcgaggccgagtct
tccgtgaaggagaaggacatgaccaaggagttcttcgagaacaagtccga
gacatggccagtgggagagagcccaaagggagtggaggagggctctatcg
ggaaggtgtgccggaccctgctggccaagagcgtgttcaactctctgtac
gcctccccacagctggagggcttcagcgccgagtctaggaagctgctgct
gatcgtgcaggccctgagagacaacctggagccagggaccttcgacctgg
gagggctgtacgaggccatcgaggagtgcctgatcaacgacccctgggtg
ctgctgaacgccagctggttcaactctttcctgacacacgccctgagatg
a

SEQ ID NO: 2 is as follows:

atggacgtgaaccccacactgctgttcctgaaggtgcccgcccagaacgc
catctccaccacattcccctacaccggcgaccccccatacagccacggaa
ccgggacaggctacaccatggacacagtgaacaggacacaccagtactct
gagagagggcggtggaccacaaacaccgagacaggagctccacagctgaa
ccccatcgacggaccactgccagaggacaacgagccatccggatacgctc
agaccgactgcgtgctggaggccatggccttcctggaggagagccacccc
gggatcttcgagacctcttgcctggagacaatggaggtggtgcagcagac
ccgggtggacaagctgacacagggcaggcagacctacgactggacactga
acagaaaccagccagctgctaccgccctggccaacacaatcgaggtgttc
agatccaacggactgaccgctaacgagagcggccggctgatcgacttcct
gaaggacgtgatggagtctatgaacaaggaggagatggagatcaccacac
acttccagagaaagaggagagtgcgggacaacatgacaaagaagatggtg
acccagagaacaatcggcaagagaaagcagcggctgaacaagaggagcta
cctgatcagagccctgaccctgaacaccatgacaaaggacgctgagagag
ggaagctgaagagaagggccatcgccaccccagggatgcagatcaggggc
ttcgtgtacttcgtggagacactggccaggagcatctgcgagaagctgga
gcagtccggactgccagtgggagggaacgagaagaaggccaagctggcca
acgtggtgcggaagatgatgaccaactctcaggacacagagatcagcttc
accatcacaggggacaacacaaagtggaacgagaaccagaaccccaggat
gttcctggccatgatcacctacatcacacggaaccagcccgagtggttca
ggaacgtgctgtccatcgcccccatcatgttcagcaacaagatggccaga
ctggggaagggctacatgttcgagtctaagtccatgaagatcagaaccca
gatcccagctgagatgctggccagcatcgacctgaagtacttcaacgact
ctaccagaaagaagatcgagaagatcagaccactgctgatcgacggaaca
gccagcctgtctcccgggatgatgatgggcatgttcaacatgctgagcac
cgtgctgggggtgtctatcctgaacctgggccagaagaggcacacaaaga
ccacatactggtgggacggcctgcagagctctgacgacttcgccctgatc
gtgaacgctccaaaccacgagggcatccaggctggcgtgaacaggttcta
cagaacatgcaagctgctggggatcaacatgagcaagaagaagagctaca
tcaaccggaccggcacattcgagttcacctctttcttctaccgatacggg
ttcgtggccaacttctccatggagctgccctctttcggggtgtccggcat
caacgagagcgccgacatgtctatcggcgtgaccgtgatcaagaacaaca
tgatcaacaacgacctgggaccagctacagctcagatggccctgcagctg
ttcatcaaggactacaggtacacctaccggtgccacaggggcgacaccca
gatccagacaagacggagcttcgagatcaagaagctgtgggagcagaccc
actctaaggctggactgctggtgtccgacggaggacccaacctgtacaac
atcaggaacctgcacatccccgaggtgtgcctgaagtgggagctgatgga
cgaggactaccagggcagactgtgcaaccccctgaaccccttcgtgaacc
acaaggacatcgagtccgtgaacaacgccgtgatcatgccagctcacgga
ccagctaagaacatggagtacgacgccgtggccaccacacacagctggat
tcccaagaggaacagatccatcctgaacaccagccagaggggcatcctgg
aggacgagcagatgtaccagaagtgctgcaacctgttcgagaagttcttc
ccctccagctcttacaggagacccgtgggcatctccagcatggtggaggc
tatggtgagccgggccaggatcgacgccaggatcgacttcgagtctggga
gaatcaagaaggaggagttcaccgagatcatgaaaatctgcagcacaatc
gaggagctgcggaggcagaagtga

SEQ ID NO: 3 is as follows:

atggagaggatcaaggagctgagaaacctgatgtctcagtcccggacaag
ggagatcctgaccaagaccacagtggaccacatggccatcatcaagaagt
acaccagcggcaggcaggagaagaaccccgccctgagaatgaagtggatg
atggccatgaagtaccccatcacagccgacaagcggatcaccgagatgat
ccccgagaggaatgagcagggccagaccctgtggagcaagatgaacgacg
ccggcagcgacagagtgatggtgagccccctggccgtgacatggtggaac
agaaacggccccgtgacatctaccgtgcactaccccaaaatctacaagac
atacttcgagaaggtggagagactgaagcacggcaccttcgggccagtgc
acttccggaaccaggtgaagatcaggagacgggtggacatcaacccagga
cacgccgacctgtctgccaaggaggcccaggacgtgatcatggaggtggt
gttccccaacgaagtgggcgccagaatcctgaccagcgagtctcagctga
ccacaaccaaggagaagaaggaggagctgcaggggtgcaagatcagcccc
ctgatggtggcctacatgctggagcgggagctggtgagaaagacccggtt
cctgccagtggctggaggcaccagcagcgtgtacatcgaggtgctgcacc
tgacacagggcacctgctgggagcagatgtacacaccaggaggagaggct
agaaacgacgacgtggaccagtccctgatcatcgccgccaggaacatcgt
gaggagagctacagtgtctgccgaccccctggcctccctgctggagatgt
gccacagcacccagatcggcggcatcaggatggtgaacatcctgaggcag
aaccccacagaggagcaggccgtggacatctgcaaggccgccatgggcct
gaggatctccagctctttctccttcggcgggttcacattcaagagaacca
gcggctccagcgtgaagagagaggaggaggtgctgacagggaacctgcag
accctgaagatcagggtgcacgagggctacgaggagttcacaatggtggg
gcggagggctaccgctatcctgagaaaggccacaagacggctgatccagc
tgatcgtgagcggccgggacgagcagtctatcgccgaggccatcatcgtg
gccatggtgttcagccaggaggactgcatgatcaaggccgtgagggggga
cctgaacttcgtgaaccgggccaaccagaggctgaaccccatgcaccagc
tgctgagacacttccagaaggacgccaaggtgctgttccagaactggggc
atcgagtctatcgacaacgtgatgggcatgatcggcatcctgcccgacat
gacaccctccaccgagatgagcatgaggggcgtgagaatctccaagatgg
gggtggacgagtactcttccgccgagaagatcgtggtgagcatcgacagg
ttcctgagagtgcgggaccagagaggcaacgtgctgctgtctcccgagga
gatcagcgagacacaggggaccgagaagctgacaatcacctacagctctt
ccatgatgtgggagatcaacggccccgagtctgtgctggtgaacacctac
cagtggatcatcagaaactgggagacagtgaagatccagtggtcccagaa
ccccaccatgctgtacaacaagatggagttcgagcccttccagagcctgg
tgccaaaggccgtgaggggccagtactctgggttcgtgagaaccctgttc
cagcagatgcgggacgtgctgggcacattcgacaccgcccagatcatcaa
gctgctgcccttcgccgccgccccaccaaagcagagccggacacagttca
gctctctgaccatcaacgtgaggggctctgggatgagaatcctggtgcgg
gggaactcccccgtgttcaactacaacaagacaaccaagcggctgaccgt
gctgggcaaggacgctggaccactgaccgaggaccccgacgagggcacag
ccggggtggagagcgccgtgctgaggggcttcctgatcctggggaaggag
gacaggagatacggccccgccctgagcatcaacgagctgtctaacctggc
caagggggagaaggccaacgtgctgatcggccagggcgacgtggtgctcg
tgatgaagaggaagagaaactccagcatcctgaccgacagccagacagcc
accaagcggatcaggatggccatcaactga

SEQ ID NO: 4 is as follows:

atggccaccaagggcacaaagaggagctacgagcagatggagaccgacgg
ggagaggcagaacgccacagagatcagagcctctgtgggcaagatgatcg
acggcatcgggagattctacatccagatgtgcaccgagctgaagctgtcc
gactacgaggggagactgatccagaactctctgacaatcgagcggatggt
gctgtccgccttcgacgagaggagaaacaagtacctggaggagcacccaa
gcgccggcaaggaccccaagaagaccggaggaccaatctacagaagggtg
gacgggaagtggagacgggagctgatcctgtacgacaaggaggagatcag
gagaatctggaggcaggctaacaacggcgacgacgctaccgctgggctga
cacacatgatgatctggcactctaacctgaacgacgccacctaccagagg
acaagagccctggtgcggaccggaatggaccccaggatgtgctctctgat
gcaggggtccacactgccacggaggagcggagctgctggagctgccgtga
agggcgtggggaccatggtcatggagctgatccggatgatcaagaggggc
atcaacgacagaaacttctggcggggcgagaacgggagacggacaaggat
cgcctacgagagaatgtgcaacatcctgaagggcaagttccagaccgccg
cccagaggacaatggtggaccaggtgagggagagcaggaaccccggcaac
gccgagttcgaggacctgatcttcctggctagatccgccctgatcctgag
agggagcgtggctcacaagtcttgcctgccagcttgcgtgtacggcagcg
ccgtggcctctgggtacgacttcgagagggagggctacagcctggtgggg
atcgaccccttcagactgctgcagaactcccaggtgtacagcctgatcag
accaaacgagaacccagctcacaagagccagctggtgtggatggcctgcc
actctgccgccttcgaggacctgagggtgagctctttcatcagaggaacc
aaggtggtgccccggggcaagctgtccacaaggggggtgcagatcgccag
caacgagaacatggagaccatggagtccagcacactggagctgcggtcca
ggtactgggccatcagaacccggagcggcgggaacacaaaccagcagagg
gcctcttccggccagatcagcatccagcccaccttcagcgtgcagagaaa
cctgcccttcgaccggcccacaatcatggctgccttcaccggaaacacag
agggacggaccagcgacatgaggacagagatcatcagactgatggagagc
gccagaccagaggacgtgtctttccagggcaggggggtgttcgagctgtc
tgacgagaaggccacctcccccatcgtgccctctttcgacatgtccaacg
agggcagctacttcttcggggacaacgccgaggagtacgacaactga

It is to be emphasized that when the present invention refers to the PA, PB1, PB2 or NP gene, unless otherwise specified, it refers to any nucleotide sequence which can encode PA, PB1, PB2 or NP protein, including nucleotide sequences that have been genetically engineered with codon preferences. The Q_PA, Q_PB1, Q_PB2, and Q_NP genes specifically refer to nucleotide sequences modified according to Vero cell preference. The mutant PA, PB1, PB2 and NP genes refer to nucleotide sequences obtained by site-directed mutagenesis of introducing a stop codon into four genes in the virus rescue system.

### Host Cell

The original host cell for expressing the viral proteins of the present invention can be selected from the group consisting of Vero cells, MDCK cells, 293 cells, MRCS and other cells. Different host cells differ in their efficacy in rescuing influenza viruses. Vero cells and MDCK cells are preferred, because the rescue efficiency of these two cells is more than twice higher than that of the remaining cells (calculated as the virus titer after rescue).

### Vector

The vector of the present invention for introducing PA, PB1, PB2 and NP genes into a mammalian cell can be selected from various conventional protein expression plasmids, preferably pBudCE4.1. To express the above genes, the present invention has constructed a recombinant plasmid which simultaneously expresses the above four genes. As an alternative technical solution, the present invention has constructed a double-plasmid system, a three-plasmid system, and a four-plasmid system expressing the above four genes, wherein each plasmid can express one or more of the above four genes. According to experimental verification, the above four systems can achieve stable expression of the target proteins. As a preferred embodiment, the present invention uses pBudCE4.1, because simultaneous expression of the four foreign genes is achieved on this plasmid, which has a relatively high transfection efficiency and has achieved a higher stable integration efficiency than other vectors. As an alternative embodiment to achieve a higher transfection and integration efficiency, the present invention also uses a double-plasmid system. The so-called double- plasmid system preferably uses pcDNA3.1/Hygro(+) as the backbone to construct the pcDNA3.1_PA_PB1 vector which simultaneously expresses PA and PB1 proteins; and uses pBudCE4.1 as the backbone to construct the pBudCE4.1_Puro_NP_PB2 vector which simultaneously expresses NP and PB2 proteins. When constructing a stable cell strain, the double plasmids are simultaneously transferred into a host cell.

The nucleotide sequence of pBudCE4.1 (SEQ ID NO: 5) is as follows:

gcgcgcgttgacattgattattgactagttattaatagtaatcaattacg
gggtcattagttcatagcccatatatggagttccgcgttacataacttac
ggtaaatggcccgcctggctgaccgcccaacgacccccgcccattgacgt
caataatgacgtatgttcccatagtaacgccaatagggactttccattga
cgtcaatgggtggactatttacggtaaactgcccacttggcagtacatca
agtgtatcatatgccaagtacgccccctattgacgtcaatgacggtaaat
ggcccgcctggcattatgcccagtacatgaccttatgggactttcctact
tggcagtacatctacgtattagtcatcgctattaccatggtgatgcggtt
ttggcagtacatcaatgggcgtggatagcggtttgactcacggggatttc
caagtctccaccccattgacgtcaatgggagtttgttttggcaccaaaat
caacgggactttccaaaatgtcgtaacaactccgccccattgacgcaaat
gggcggtaggcgtgtacggtgggaggtctatataagcagagctctctggc
taactagagaacccactgcttactggcttatcgaaattaatacgactcac
tatagggagacccaagcttgcattcctgcaggtcgacatcgatcttaagc
agtacttctagaggatccgaacaaaaactcatctcagaagaggatctgaa
tatgcataccggtcatcatcaccatcaccattgagtttgatccccgggaa
ttcagacatgataagatacattgatgagtttggacaaaccacaactagaa
tgcagtgaaaaaaatgctttatttgtgaaatttgtgatgctattgcttta
tttgtaaccattataagctgcaataaacaagttggggtgggcgaagaact
ccagcatgagatccccgcgctggaggatcatccagccggcgtcccggaaa
acgattccgaagcccaacctttcatagaaggcggcggtggaatcgaaatc
tcgtagcacgtgtcagtcctgctcctcggccacgaagtgcacgcagttgc
cggccgggtcgcgcagggcgaactcccgcccccacggctgctcgccgatc
tcggtcatggccggcccggaggcgtcccggaagttcgtggacacgacctc
cgaccactcggcgtacagctcgtccaggccgcgcacccacacccaggcca
gggtgttgtccggcaccacctggtcctggaccgcgctgatgaacagggtc
acgtcgtcccggaccacaccggcgaagtcgtcctccacgaagtcccggga
gaacccgagccggtcggtccagaactcgaccgctccggcgacgtcgcgcg
cggtgagcaccggaacggcactggtcaacttggccatggtttagttcctc
accttgtcgtattatactatgccgatatactatgccgatgattaattgtc
aacacgtgctgatcagatccgaaaatggatatacaagctcccgggagctt
tttgcaaaagcctaggcctccaaaaaagcctcctcactacttctggaata
gctcagaggcagaggcggcctcggcctctgcataaataaaaaaaattagt
cagccatggggcggagaatgggcggaactgggcggagttaggggcgggat
gggcggagttaggggcgggactatggttgctgactaattgagatgcatgc
tttgcatacttctgcctgctggggagcctggggactttccacacctggtt
gctgactaattgagatgcatgctttgcatacttctgcctgctggggagcc
tggggactttccacaccctcgtcgagctagcttcgtgaggctccggtgcc
cgtcagtgggcagagcgcacatcgcccacagtccccgagaagttgggggg
aggggtcggcaattgaaccggtgcctagagaaggtggcgcggggtaaact
gggaaagtgatgtcgtgtactggctccgcctttttcccgagggtggggga
gaaccgtatataagtgcagtagtcgccgtgaacgttctttttcgcaacgg
gtttgccgccagaacacaggtaagtgccgtgtgtggttcccgcgggcctg
gcctctttacgggttatggcccttgcgtgccttgaattacttccacctgg
ctccagtacgtgattcttgatcccgagctggagccaggggcgggccttgc
gctttaggagccccttcgcctcgtgcttgagttgaggcctggcctgggcg
ctggggccgccgcgtgcgaatctggtggcaccttcgcgcctgtctcgctg
ctttcgataagtctctagccatttaaaatttttgatgacctgctgcgacg
ctttttttctggcaagatagtcttgtaaatgcgggccaggatctgcacac
tggtatttcggtttttgggcccgcggccggcgacggggcccgtgcgtccc
agcgcacatgttcggcgaggcggggcctgcgagcgcggccaccgagaatc
ggacgggggtagtctcaagctggccggcctgctctggtgcctggcctcgc
gccgccgtgtatcgccccgccctgggcggcaaggctggcccggtcggcac
cagttgcgtgagcggaaagatggccgcttcccggccctgctccagggggc
tcaaaatggaggacgcggcgctcgggagagcgggcgggtgagtcacccac
acaaaggaaaagggcctttccgtcctcagccgtcgcttcatgtgactcca
cggagtaccgggcgccgtccaggcacctcgattagttctggagcttttgg
agtacgtcgtctttaggttggggggaggggttttatgcgatggagtttcc
ccacactgagtgggtggagactgaagttaggccagcttggcacttgatgt
aattctcgttggaatttgccctttttgagtttggatcttggttcattctc
aagcctcagacagtggttcaaagtttttttcttccatttcaggtgtcgtg
aacacgtggtcgcggccgcttcgaaggtaccagcacagtggactcgagag
atctggccggctgggcccgtttcgaaggtaagcctatccctaaccctctc
ctcggtctcgattctacgcgtaccggtcatcatcaccatcaccattgagt
ttaaacccgctgatcagcctcgactgtgccttctagttgccagccatctg
ttgtttgcccctcccccgtgccttccttgaccctggaaggtgccactccc
actgtcctttcctaataaaatgaggaaattgcatcgcattgtctgagtag
gtgtcattctattctggggggtggggtggggcaggacagcaagggggagg
attgggaagacaatagcaggcatgctggggatgcggtgggctctatggct
tctgaggcggaaagaaccagtggcggtaatacggttatccacagaatcag
gggataacgcaggaaagaacatgtgagcaaaaggccagcaaaaggccagg
aaccgtaaaaaggccgcgttgctggcgtttttccataggctccgcccccc
tgacgagcatcacaaaaatcgacgctcaagtcagaggtggcgaaacccga
caggactataaagataccaggcgtttccccctggaagctccctcgtgcgc
tctcctgttccgaccctgccgcttaccggatacctgtccgcctttctccc
ttcgggaagcgtggcgctttctcatagctcacgctgtaggtatctcagtt
cggtgtaggtcgttcgctccaagctgggctgtgtgcacgaaccccccgtt
cagcccgaccgctgcgccttatccggtaactatcgtcttgagtccaaccc
ggtaagacacgacttatcgccactggcagcagccactggtaacaggatta
gcagagcgaggtatgtaggcggtgctacagagacttgaagtggtggccta
actacggctacactagaaggacagtatttggtatctgcgctctgctgaag
ccagttaccttcggaaaaagagttggtagctcttgatccggcaaacaaac
caccgctggtagcggtggtttttttgtttgcaagcagcagattacgcgca
gaaaaaaaggatctcaagaagatcctttgatcttttctacggggtctgac
gctcagtggaacgaaaactcacgttaagggattttggtcatgacattaac
ctataaaaataggcgtatcacgaggccctttcgtctcgcgcgtttcggtg
atgacggtgaaaacctctgacacatgcagctcccggagacggtcacagct
tgtctgtaagcggatgccgggagcagacaagcccgtcagggcgcgtcagc
gggtgttggcgggtgtcggggctggcttaactatgcggcatcagagcaga
ttgtactgagagtgcaccatatatgcggtgtgaaataccgcacagatgcg
taaggagaaaataccgcatcaggcgccattcgccattcaggctgcgcaac
tgttgggaagggcgatcggtgcgggcctcttcgctattacgcca

### Influenza Virus Rescue System

Virus rescue, also known as infectious molecular cloning of virus, belongs to a reverse genetic manipulation technique in which cells are transfected under certain conditions with a suitable form of viral nucleic acid by manually manipulating the genes to produce infectious virions. The present invention optionally uses the eight-plasmid rescue system established by Hoffmann et al., the advantage of a completely plasmid-based system is that it does not require a helper virus and avoids a lot of screening work. The eight-plasmid system is based on the least number of plasmids, and the same vector is used to realize the synthesis of viral RNA and proteins in the cells, and then they are packaged into a virus. The eight-plasmid system of the present invention can be, for example, pPolI-M-PB2, pPolI-M-PB1, pPolI-M-PA, pPolI-M-NP, pPolI-WSN-HA, pPolI-WSN-NA, pPolI-WSN-M, pPolI-WSN-NS. Among them, the carried PA, PB1, PB2 and NP genes are modified by the mutation of the mutation sites selected by the present invention. The recombinant plasmids are constructed using the PHH21 vector. After completion of the construction, the recombinant plasmid can be obtained completely by artificial synthesis.

### Site-Directed Mutagenesis

The present invention conducts a site-directed mutagenesis of introducing a stop codon into four genes (PA, PB1, PB2 and NP) in a virus rescue system, the purpose of which is to enable the rescue system to achieve virus rescue and infection proliferation in the cell line constructed above, whereas in normal animal cells, due to the introduction of the stop codon, PA, PB1, PB2 and NP could not be expressed normally, and thus the virus rescue and infection proliferation cannot be achieved. Among the four genes, PA, PB1 and PB2 respectively encode the three subunits of the RNA polymerase complex, and NP encodes a nuclear protein, the co-inactivation of the four genes ensures that the virus cannot replicate and proliferate in normal cells, i.e., produces replication-controllable traits. The methods of site-directed mutagenesis include but are not limited to the introduction of a TAG codon into four gene sequences, respectively, resulting in termination of expression. As to the mutation sites, a series of the most efficient mutation sites can be screened by analyzing the amino acid sequence and crystal structure of the protein. Effective mutations can be, for example, PA (R266 is mutated to TAG), PB1 (R52 is mutated to TAG), PB2 (K33 is mutated to TAG), and NP (D101 is mutated to TAG). In addition, mutations in the HA, NA, M and NS genes can additionally be included.

### Cell Electrotransformation

The present invention uses the method of electrotransformation to transfect PA, PB1, PB2 and NP genes into Vero, MDCK or other cells. Experiments show that the electrotransformation method is superior to other methods, and Vero cells and MDCK cells are most suitable.

### Safety and Effectiveness

A method for identifying safety-replication defects is a cytopathic test. In vitro and in vivo experiments have shown that replication-controllable influenza viruses cannot replicate in wild-type animal cells, but can be rescued to form intact live viruses in host cells that have expressed PA, PB1, PB2 and NP proteins, resulting in cytopathies and cytolysis. Through cytopathies, it can be known whether the influenza virus has the ability to replicate in different cells. Viral immunity can be assessed by routine animal immunization experiments. Experiments have shown that the live virus of the present invention has very high safety and genetic stability, and has a better immune effect comparing to inactivated virus.

### Immunogenic Composition

The present invention also provides an immunogenic composition (e.g., a vaccine) comprising a live virus obtained by the rescue method of the present invention. In some embodiments, the live virus is attenuated. In some embodiments, the immunogenic composition comprises two, three, four or more types of live viruses. The specification of the vaccine can be, for example, a live influenza virus containing 10^{6.5}-10^{7.5} FFU per dose. In certain embodiments, the immunogenic composition (e.g., a vaccine) is packaged as a pre-filled spray containing, for example, a single dose of 0.2 ml. The pharmaceutically acceptable medium of the vaccine of the present invention is preferably an aqueous solution or emulsion. More preferably, a water-in-oil emulsion medium is used. The specific formulation of the vaccine will depend on the viral vector used, as well as the inserted foreign nucleotide sequence.

In certain embodiments, the composition described herein comprises an adjuvant or is administered in combination with an adjuvant. Adjuvants administered with the composition described herein can be administered prior to, concurrently with, or subsequent to administration of the composition. In some embodiments, the term “adjuvant” refers to a compound that enhances, improves and/or strengthens the immune response to the influenza virus vaccine when administered in combination with or as part of the composition described herein, however, does not produce an immune response to the polypeptide when administered alone.

### Preventive and Therapeutic Use

The influenza virus of the present invention can be used for the preparation of vaccines and for preventive or therapeutic use. The effective dose of the pharmaceutical composition depends on the nature of the disease or condition and can be determined by standard clinical techniques. In addition, in vitro test can be used to help determine the optimal dose range. The precise dose to be employed in the formulation will also depend on the route of administration, and the severity of the disease or condition, and should be determined by the physician's judgment and the condition of each patient. However, a suitable dosage range for administration will generally be about 10⁴-5×10⁷ pfu, administered once, or multiple doses as needed. The pharmaceutical composition of the present invention contains 10⁴-5×10⁷ pfu of a mutant replication-controllable virus which can be administered intranasally, intratracheally, intramuscularly or subcutaneously. An effective dose can be derived from the dose response curve, which can be derived from an in vitro or animal model test system.

The innovation of the present invention lies in:

it provides a brand new method for producing a mutant influenza virus, and the influenza virus produced by the method can be rescued, infect and proliferate only in a specific cell line, but will not replicate and proliferate in normal animal and human cells; the present invention greatly improves the safety of the virus while retaining the extremely high immunogenicity as a live virus and provides a new choice for the prevention and treatment of human influenza viruses; using the virus rescue system of the present invention, the inventors have unexpectedly found that viral rescue, infection and proliferation can be achieved in Vero or MDCK cells integrated with foreign viral proteins, and verified the activity of the assembled virus for the first time.

The present invention uses codon-optimized foreign viral genes to cooperate with Vero cells or MDCK cells to achieve optimal expression of the foreign proteins. At the same time, the preferred vector ensures that the four genes are simultaneously transferred into the host cells and stably passaged and expressed in the cells, and the cells can be used not only as an essential host cell for virus rescue, but also be used as a companion cell matrix for the replication-controllable viruses for mass production of viruses or vaccines.

### DESCRIPTION OF THE DRAWINGS

FIG. 1 is a map of the constructed plasmid pBudCE4.1_NP_IRES_PA_PB2_IRES_PB1.

FIG. 2 is a map of the constructed plasmid pcDNA3.1_PA_PB1 of the double-plasmid system.

FIG. 3 is a map of the constructed plasmid pBudCE4.1_Puro_NP_PB2 of the double-plasmid system.

FIG. 4 is an electron micrograph of the virus formed by rescue (transmission electron microscope (Spirit 120 KV), 100 kv, magnified 150,000 times).

FIG. 5 is the sequencing result of the verification of the mutation sites of the virus produced by rescue, wherein the gene loci 1-4 correspond to the corresponding loci of NP, PA, PB1 and PB2, respectively, and in the comparison map of the same gene locus, the upper sequence is the measured sequence, and the lower sequence is the reference sequence.

FIG. 6 is the safety comparison experiment for transfected cells for rescuing the virus, wherein A1 is the electron micrograph of the transfected normal cells after plating overnight, A2 is the electron micrograph of the transfected normal cells after 3 days of culture; B1 is the electron micrograph of the transfected cells with the single-plasmid constructed according to Example 1 after plating overnight, B2 is the cell electron micrograph after 3 days of culture. The virus solutions used was obtained by using the cells of Example 3-1 to participate in rescue.

### DETAILED DESCRIPTION OF THE INVENTION

The technical solutions in the examples of the present invention are clearly and completely described below. It is obvious that the described examples are only a part of the examples of the present invention, but not all of the examples. All other examples obtained by those of ordinary skill in the art based on the examples of the present invention without paying creative work are within the protection scope of the present invention.

It should be noted that the terms “comprising” and “having” and any of their variations of the examples of the present invention are intended to cover a non-exclusive inclusion, for example, a process, method, system, product or device that comprises a series of steps or units which are not necessarily limited to those clearly listed steps or units, but may include other steps or units not clearly listed or inherent to those processes, methods, products or devices.

### EXPERIMENTAL MATERIALS

The codon-optimized NP, PA, PB1 and PB2 gene sequences of the present invention are all derived from artificial synthesis, and all primers for sequence cloning and selectable markers are also artificially synthesized according to the sequences disclosed in the present invention. The vector pBudCE4.1 (SEQ ID NO: 5) was synthesized artificially.

The host cells in the examples are Vero cells from ATCC® CCL-81™ and belong to commercially available Vero cell line.

### Example 1 Construction of Plasmid pBudCE4.1_NP_IRES_PA_PB2_IRES_PB1

According to the gene sequence of influenza virus A/WSN/1933 (Taxonomy ID: 382835) published by Pubmed, the expression gene sequences of PA, PB1, PB2 and NP which are preferred for Vero cells were obtained by codon optimization, respectively. After the whole gene synthesis, the obtained gene fragments were named as Q_PA (SEQ ID NO: 1), Q_PB1 (SEQ ID NO: 2), Q_PB2 (SEQ ID NO: 3) and Q_NP (SEQ ID NO: 4) genes, respectively.

pBudCE4.1 and Q PURO (puromycin resistance gene, SEQ ID NO: 6) were double-digested (EcoRI, AvrII), and ligated using T4 DNA ligase. The resulting plasmid was transformed into E. coli DH5a by heat shock, screened, cultured and amplified, and purified to obtain pBudCE4.1+puro.

pBudCE4.1+puro and Q_NP were double-digested (HindIII, SalI), and ligated using T4 DNA ligase. The resulting plasmid was transformed into E. coli DH5a by heat shock, screened, cultured and amplified, and purified to obtain pBudCE4.1+puro_NP.

pBudCE4.1+puro_NP and Q_PA were double-digested (SalI, BamHI), and ligated using T4 DNA ligase. The resulting plasmid was transformed into E. coli DH5a by heat shock, screened, cultured and amplified, and purified to obtain pBudCE4.1 +puro_NP_PA.

pBudCE4.1+puro_NP_PA and Q_PB2 were double-digested (NotI, XhoI), and ligated using T4 DNA ligase. The resulting plasmid was transformed into E. coli DH5a by heat shock, screened, cultured and amplified, and purified to obtain pBudCE4.1+puro_NP_PA_PB2.

pBudCE4.1+puro_NP_PA_PB2 and Q_PB1 were double-digested (XhoI, BglII), and ligated using T4 DNA ligase. The resulting plasmid was transformed into E. coli DH5a by heat shock, screened, cultured and amplified, and purified to obtain pBudCE4.1+puro_NP_PA_PB2_PB1.

In the above construction process, the solid medium used in the screening steps was: low sodium LB solid medium (1% peptone, 0.5% sodium chloride, 0.5% yeast extract, 1.8% agarose) containing 25 μg/ml bleomycin. The liquid medium used in the amplification steps was: low sodium LB liquid medium (1% peptone, 0.5% sodium chloride, 0.5% yeast extract) containing 25 μg/ml bleomycin.

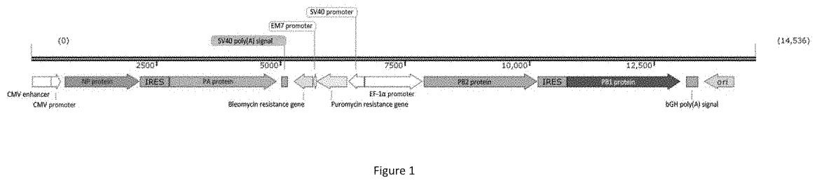

The map of this constructed plasmid is shown in FIG. 1. The nucleotide sequence of the Q PURO gene (SEQ ID NO: 6) is as follows:

atgaccgagtacaagcccacggtgcgcctcgccacccgcgacgacgtccc
ccgggccgtacgcaccctcgccgccgcgttcgccgactaccccgccacgc
gccacaccgtcgacccggaccgccacatcgagcgggtcaccgagctgcaa
gaactcttcctcacgcgcgtcgggctcgacatcggcaaggtgtgggtcgc
ggacgacggcgccgcggtggcggtctggaccacgccggagagcgtcgaag
cgggggcggtgttcgccgagatcggcccgcgcatggccgagttgagcggt
tcccggctggccgcgcagcaacagatggaaggcctcctggcgccgcaccg
gcccaaggagcccgcgtggttcctggccaccgtcggcgtctcgcccgacc
accagggcaagggtctgggcagcgccgtcgtgctccccggagtggaggcg
gccgagcgcgccggggtgcccgccttcctggagacctccgcgccccgcaa
cctccccttctacgagcggctcggcttcaccgtcaccgccgacgtcgagg
tgcccgaaggaccgcgcacctggtgcatgacccgcaagcccggtgcctag

### Example 2 Construction of Double-Plasmid System

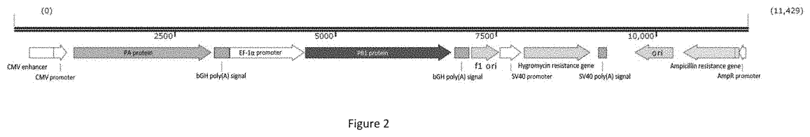

The construction process of pcDNA3.1_PA_PB1 vector is shown in FIG. 2, and is specifically as follows: pcDNA3.1/Hygro(+) plasmid (purchased from Biofeng, source: Invitrogene) and the vector (pUC57, purchased from Biofeng) containing PA protein gene (Q_PA,synthesized by GenScript) (the vector was obtained by double digesting the PA gene and pUC57 with HindIII and BamHI and ligating them for amplification) were simultaneous subjected to double digestion with HindIII and BamHI and 1% agarose gel electrophoresis. pcDNA3.1/Hygro(+) plasmid and PA protein gene fragment (Q_PA) were recovered from the gels and the above recovered fragments were ligated using T4 DNA ligase (the molar ratio of the vector to the insert was 1:3) and transformed into E. coli DH5a by heat shock method to construct pcDNA3.1_PA plasmid.

The pcDNA3.1_PA plasmid and the vector (pUC57, purchased from Biofeng) containing the PB1 protein gene (Q_PB1, synthesized by GenScript) (the vector was obtained by double digesting the PB1 gene and pUC57 with HindIII and BamHI and ligating them for amplification) were simultaneous subjected to double digestion with BamHI and NotI and 1% agarose gel electrophoresis. pcDNA3.1_PA plasmid and PB1 protein gene fragment (Q_PB1) were recovered from the gels and the above recovered fragments were ligated using T4 DNA ligase (the molar ratio of the vector to the insert was 1:3) and transformed into E. coli DH5a by heat shock method to construct pcDNA3.1_PA_PB1 plasmid.

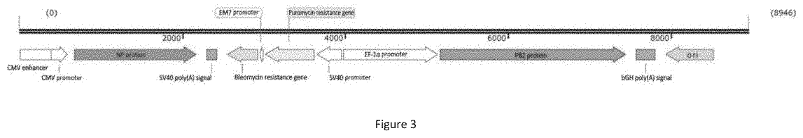

The construction process of pBudCE4.1_Puro_NP_PB2 vector expressing NP and PB2 proteins simultaneously with pBudCE4.1 as the backbone can be referred to Example 1, except that the PB2 gene was directly introduced after introduction of the NP gene. The map of this constructed plasmid is shown in FIG. 3.

### Example 3 Vero Cell Culture and Electrotransformation

### Example 3-1 Electrotransformation of Vero Cells with Single-Plasmid

The thawed Vero cells were transferred to a T25 cell culture flask at a cell concentration of 9×10⁶ cells/vial, and the cell culture medium used was MEM containing L-glutamine, non-essential amino acids and 10% FBS. On the second day of culture, after trypsinization, the cells were collected and resuspended in 50 ml of PBS, centrifuged at 4000 rpm for 10 min, and resuspended in 0.5 mL of optiMEM (purchased from Sigma, USA). The cells were counted by FCM (1:40), and every 5×10⁶ cells were added to a 0.4 cm electroporation cuvette containing 400 μL of optiMEMI (purchased from Sigma, USA).

1 μg of plasmid pBudCE4.1+puro_NP_PA_PB2_PB1 was transferred into Vero cells (1.67E+04 cells/μg plasmid) by electrotransformation. The electrotransformation conditions were: voltage: 120 V, pulse: 500 μs, number of electric shocks: 6 times, time interval: 100 ms.

### Example 3-2 Electrotransformation of MDCK Cells with Single-Plasmid

As another alternative solution, MDCK cells were seeded in a T75 cell culture flask at a concentration of 1.0×10⁶ cells/vial, and the cell culture medium used was MEM containing 4 mmol/L L-glutamine, non-essential amino acids and 10% FBS. On the second day of culture, the cells were collected and resuspended in 5 ml of MEM, counted, centrifuged, and washed by adding 3 mL of EK buffer, centrifuged again, and resuspended in EK buffer to a cell density of 2.5E+06 cells/mL, and transferred to a 96 well-plate with 60 μL in each well. 9 μg of plasmid (1.2 μg/μL, 4.1 μL) pBudCE4.1+puro_NP_PA_PB2_PB1 was transferred into MDCK cells in the well by electrotransformation. The electrotransformation conditions were: voltage: 175 V, pulse: 100 μs, number of electric shocks: 6 times, time interval: 1000 ms.

### Example 3-3 Double-Plasmid Electrotransformation

The Vero cells were seeded in a T75 cell culture flask at a concentration of 1.0×10⁶ cells/vial, and the cell culture medium used was DMEM containing 4 mmol/L L-glutamine, non-essential amino acids and 20% FBS DMEM. On the second day of culture, the cells were collected and resuspended in 5 ml of DMEM, counted, centrifuged, and washed by adding 3 mL of EK buffer, centrifuged again, and resuspended in EK buffer to a cell density of 2.5E+06 cells/mL, and transferred to a 96 well-plate with 60 μL in each well. 5 μg of plasmid (1.2 μg/μL, 4.1 μL) pcDNA3.1_PA_PB1 and 5 μg of plasmid pBudCE4.1_Puro_NP_PB2 were transferred into Vero cells in the wells by electrotransformation, respectively. The electrotransformation conditions were: voltage: 175 V, pulse: 100 μs, number of electric shocks: 6 times, time interval: 1000 ms.

### Example 3-4 Culture after Electrotransformation

After electrotransformation, 3-5 cell pools with better growth conditions were taken and the culture was enlarged.

Vero cell culture conditions: 250 μg/ml hygromycin, 2 μg/ml puromycin, and the cell culture medium was DMEM containing 4 mmol/L L-glutamine, non-essential amino acids and 20% FBS. The culture was statically maintained at 37° C., 5% carbon dioxide; the medium was changed every 2 days.

MDCK cell culture conditions: 100 μg/ml hygromycin, 0.5 μg/ml puromycin, and the cell culture medium was DMEM containing 4 mmol/L L-glutamine, non-essential amino acids and 20% FBS. The culture was statically maintained at 37° C., 5% carbon dioxide; the medium was changed every 2 days.

### Example 3-5 RT-PCR

The total RNA in the above cultured cells was extracted by TRIzol, and reverse-transcribed into cDNA by M-MLV reverse transcriptase using random primers, and the expression of the target gene was detected by PCR amplification using the reverse transcribed cDNA as a template. RT-PCR results demonstrated stable expression of mRNA levels of PA, PB2, PB1 and NP genes in single-plasmid or double-plasmid transformed Vero cells and single-plasmid transformed MDCK cells.

### Example 4 Construction of Mutant Virus RNA Rescue System

According to the gene sequence of influenza virus A/WSN/1933 (Taxonomy ID: 382835) published by Pubmed, the genes of the eight gene fragments of the influenza virus were obtained by whole gene synthesis. The GeneBank accession numbers of the influenza virus gene sequences are: PB2: LC333182.1, PB1: LC333183.1, PA: LC333184.1, NP: LC333186.1, HA: LC333185.1, NA: LC333187.1, M: LC333188.1, NS: LC333189.1, respectively. Then, they were respectively ligated to the vector PHH21 (purchased from Biovector Science Lab, Inc.) to obtain plasmids for rescuing the wild-type influenza virus WSN. The obtained plasmids were named: pPolI-WSN-PB2, pPolI-WSN-PB1, pPolI-WSN-PA, pPolI-WSN-NP, pPolI-WSN-HA, pPolI-WSN-NA, pPolI-WSN-M and pPolI-WSN-NS, respectively. The method of constructing the 8-plasmid system is an existing technology, and can also be constructed and used with reference to the related patent documents, for example, the construction method in Chinese Patent Application No. 201511029463.1.

The amino acid conservation of the influenza virus A/WSN/1933 proteins was analyzed by the bioinformatics tool, Consurf, and based on the crystal structures of the influenza virus proteins that have been resolved, conservative, relatively conservative, relatively non-conservative, non-conservative amino acid sites were selected for mutation, and selectable mutations for PA, PB1, PB2 and NP were finally screened and obtained, i.e., PA (R266 codon was mutated to TAG), PB1 (R52 codon was mutated to TAG), PB2 (K33 codon was mutated to TAG) and NP (D101 codon was mutated to TAG) mutations.

For the above selected mutation sites, primers capable of mutating the codons encoding the above amino acids to TAG were designed, and the specific primers are as follows:

TABLE 1
Point Mutation Primers for Selected Sites
on Four Proteins

| | | SEQ | |
|---|---|---|---|
| Primer | | ID | |
| Name | Primer Sequence (5′-3′) | NO: | Site |
| NP | 5′-ggacctatatacaggagagtatag | SEQ | D101 |
| | ggaaagtggaggagaga-3′ | ID | |
| | | NO: | |
| | | 7 | |
| | 5′-tctctcctccactttccctatact | SEQ | |
| | ctcctgtatataggtcc-3′ | ID | |
| | | NO: | |
| | | 8 | |
| PA | 5′-gcccatccggaagtctaagtggct | SEQ | R266 |
| | atggtgttgatttcaaaaaaggttca-3′ | ID | |
| | | NO: | |
| | | 9 | |
| | 5′-tgaaccttttttgaaatcaacacca | SEQ | |
| | tagccacttagacttccggatgggc-3′ | ID | |
| | | NO: | |
| | | 10 | |
| PB2 | 5′-ctgtcttcctgatgtgtactacttg | SEQ | K33 |
| | attatggccatatg-3′ | ID | |
| | | NO: | |
| | | 11 | |
| | 5′-catatggccataatcaagtagtacac | SEQ | |
| | atcaggaagacag-3′ | ID | |
| | | NO: | |
| | | 12 | |
| PB1 | 5′-gtttgttgtccatcttccctattct | SEQ | |
| | gagtactgatgtgtcct-3′ | ID | |
| | | NO: | |
| | | 13 | |
| | 5′-aggacacatcagtactcagaatagg | SEQ | R52 |
| | gaagatggacaacaaac-3′ | ID | |
| | | NO: | |
| | | 14 | |

Using pPolI-WSN-PB2, pPolI-WSN-PB1, pPolI-WSN-PA, pPolI-WSN-NP as template plasmids and the site-directed mutagenesis kit (Lightning Site-Directed Mutagenesis Kits Catalog#210518), the amino acid codon of the selected sites on each protein was mutated to the amber stop codon TAG by the above primer sites according to the instructions, and the mutations were successful as verified by sequencing. The resulting vectors containing the mutant genes were named as pPolI-M-PB, pPolI-M-PB1, pPolI-M-PA and pPolI-M-NP, respectively.

### Example 5 Rescue of Replication-Controllable Virus

The cells stably expressing genes prepared in Example 3-1 were plated: using a 10 cm dish as an example, the stable strain was plated in a 10 cm dish plate at 1×10⁶ cells/ml per dish, mixed, and placed at 37° C., 5% CO₂, cultured for 20-24 hrs to reach 20% -40% cell confluence.

The transfection of the viral plasmid and virus rescue were carried out in accordance with a conventional method, and the eight plasmids used to rescue the influenza virus were co-transfected into Vero stable cell lines transformed with single-plasmid or double -plasmid obtained in Example 3. Among them, the plasmids carrying the NP, PA, PB2 and PB1 genes were the four site-directed mutant plasmids obtained in Example 4, and the plasmids carrying the HA, NA, M and NS genes were the four plasmids: pPolI-WSN-HA, pPolI-WSN-NA, pPolI-WSN-M and pPolI-WSN-NS.

1 ml of Opti-MEM was added to a EP tube, eight plasmids (1 μg per plasmid) were added, and transfection reagent was added, mixed, incubated at 37° C., 5% CO₂. After 5.5 hours of culture, the medium was changed, half of the medium was changed on the 4th day after transfection, and 10% of the initial volume was supplemented every two days from the 6th day after transfection. The growth of the cells and the pathological changes of the cells were observed and recorded every day. In the virus rescue involving the stable cell lines produce by single-plasmid or double-plasmid transformation, more than 70% have pathological changes for both of them and viral solutions were harvested.

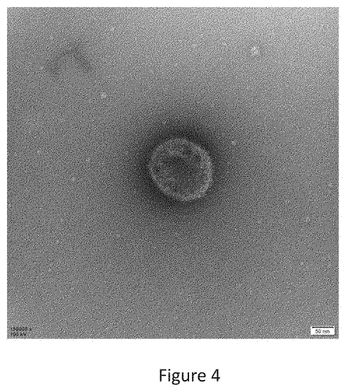

The viruses were negatively stained with phosphotungstic acid for 5 min, and observed by transmission electron microscopy (Spirit 120 KV) (100 kv, magnified 150,000 times). A complete virus image obtained by the rescue involving the single-plasmid transformed Vero cells was photographed, as shown in FIG. 4.

The same results were obtained with the double-plasmid-integrated Vero cells as well as the stably integrated MDCK cells.

### Example 6 Confirmation of Mutation Sites of Replication-Controllable Virus

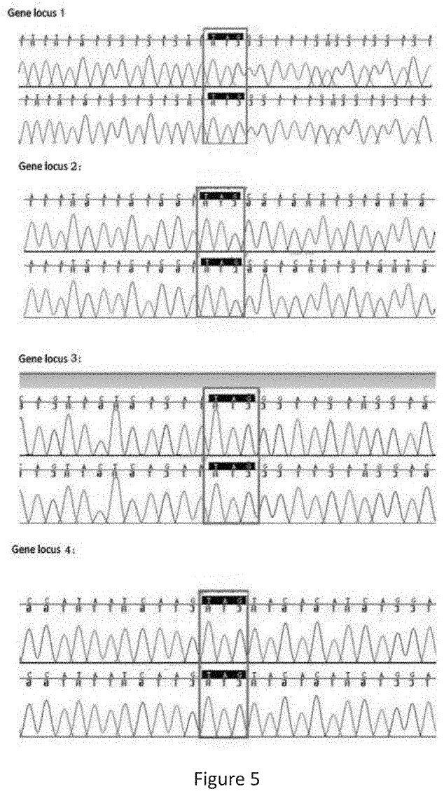

The total RNA of the virus obtained in Example 5 (rescue involving the single plasmid-integrated Vero cell line) was extracted using RNAiso plus (TaKaRa Code No.: 9108), and after extraction, it was dissolved in 13 μl of RNase-free dH₂O, and TaKaRaPrimeScript™ One Step RT-PCR Kit Ver. 2 (Code No.: RR055A) was used for RT-PCR, TSINGKE DNA Gel Recovery Kit (Code No.: GE0101-200) was used to recover the above PCR product, and the PCR product was dissolved in 25 μl of Eluent Buffer and subjected to BigDye®Terminator v3.1 sequencing reaction and purification, 3730 sequencer sequencing, and the 3730×1 was used to collect and analyze the data. See FIG. 5 for the results. The primers for the four mutation sites are as follows:

TABLE 2

| | Primer sequence | |
|---|---|---|
| Gene | (5′-3′) | Length |
| NP | GATGGAGAACGCCAGAAT | 18 |
| (Gene | (SEQ ID NO: 15) | |
| locus 1) | GCATCGTCACCATTATTAGC | |
| | (SEQ ID NO: 16) | 20 |
| PA | ACCAGGCTATTCACCATAAG | |
| (Gene | (SEQ ID NO: 17) | 20 |
| locus 2) | CCTTCCATCCAAAGAATGTTC | |
| | (SEQ ID NO: 18) | 21 |
| PB1 | GGACAGGAACAGGATACAC | |
| (Gene | (SEQ ID NO: 19) | 19 |
| locus 3) | TGCTGAACAACCTCCATC | |
| | (SEQ ID NO: 20) | 18 |
| PB2 | CGAAAGCAGGTCAATTATATTCA | |
| (Gene | (SEQ ID NO: 21) | 23 |
| locus 4) | TACTTGTCACTGGTCCATTC | |
| | (SEQ ID NO: 22) | 20 |

Experimental results: as shown in FIG. 5, the sequencing results of the four mutation sites were completely consistent with the reference sequences, and no back mutation occurred. The mutation detection was also performed for the virus rescue in which the host cells constructed by the double-plasmid system were involved, and the results were the same.

### Example 7 Confirmation of Safety by Viral Cytopathic Experiment

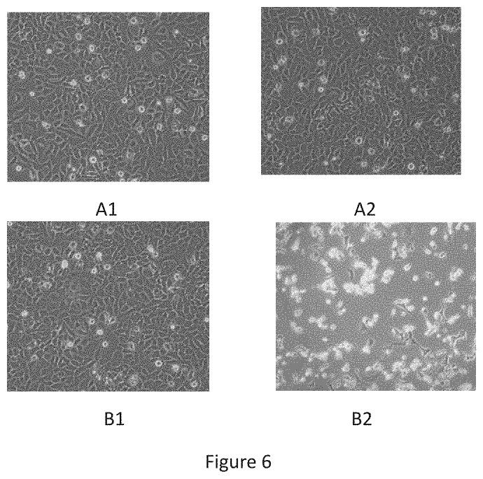

The normal Vero cells and the cells constructed in Example 3-1 were taken and plated in 6-well plates at 4*10⁵ cells/well, and 2 mL of the culture medium was added to each well. The cells were cultured at 37° C., 5% CO₂ overnight, and then 0.5 mL of virus solution was added (prepared in Example 5) and mixed. The mixture was cultured at 37° C., 5% CO₂ for 3 days, observed under the microscope. The constructed cells were lysed due to viral replication, and normal Vero cells were not lysed. See FIG. 6.

### Example 8 Determination of HA by Virus Hemagglutination Test

The replication-controllable virus solution obtained in Example 5 and the virus solution obtained by virus rescue involving MDCK prepared by the method taught in the examples of the present application were taken and divided into three groups of 6 samples for detection. Among them, the samples 1 and 2 were respectively the virus solutions (cultured for 6 days and 7 days, respectively) formed by the rescue involving the cells of Example 3-1, and the samples 3 and 4 were respectively the virus solutions (cultured for 6 days and 7 days, respectively) formed by the rescue involving the cells of Example 3-2, and the samples 5 and 6 were respectively the virus solutions (cultured for 6 days and 7 days, respectively) formed by the rescue involving the cells of Example 3-3. The red blood cell suspension was prepared according to the “1% chicken red blood cell suspension preparation” SOP. The microplate were placed horizontally: the vertical directions were called the columns, for example, the holes A1 to H1 were called the first column; the parallel directions were called the rows, for example, A1 to A12 were called row A. The laboratory number and sequence of loading samples of the virus to be tested were labeled. Adding PBS: an 8-channel sampler was taken and equipped with a 200 μL filter-type dripper; 50 μL of PBS was pipetted from the loading slot and added into the second column of the microplate and 50 μL of PBS was added in sequence until the last column. Adding the virus to be tested: a single-channel pipette was equipped with a 200 μL filter-type dripper, and 100 μL of the virus solution to be tested was pipetted and added to the labeled corresponding well of the first column of the microplate. 100 μL of PBS was added to the last H1 well as a red blood cell control. The 8-channel sampler was equipped with a 200 μL filter-type dripper. 50 μL of virus solution was taken from each well of the first column, and added to the corresponding wells of the second column, and mixed several times. Two-fold serial dilutions were sequentially performed from the second column to the twelfth column of the microplate. In the last column, 50 μL of liquid was discarded per well. The 8-channel sampler was equipped with a 200 μL filter-type dripper and 50 μL of red blood cell suspension was taken from the loading slot. 50 μL of 1% red blood cell suspension was added to each well and the microplate was flicked to mix the red blood cells with the virus. The plate was incubated for 60 minutes at room temperature, the red blood cell agglutination phenomenon was observed and the results were recorded. Determination of results: the determination of the red blood cell agglutination titer was determined by the highest dilution at which the complete agglutination occurred, and the reciprocal of the dilution was the red blood cell agglutination titer of the virus. Complete red blood cells agglutination was record as “+”; no agglutination or partial agglutination was recorded as “−”. The blank control was conducted by replacing 50 μL of virus solution with 50 μL of PBS. The positive control was a wild-type influenza virus A/WSN/1933 sample diluted 100 times by PBS. The negative control 1 was Vero cells that were not transformed with a plasmid or plasmid system containing pBudCE4.1+puro_NP_PA_PB2_PB1 and other plasmids, but transiently transformed with the 8-plasmid system containing the mutations; the negative control 2 was Vero cells that were not transformed with the 8-plasmid system containing the mutations, but transformed with the pBudCE4.1+puro_NP_PA_PB2_PB1 vector.

TABLE 3
Red Blood Cell Agglutination

| | Hemagglutination titer | | | |
|---|---|---|---|---|
| Sample number | 1:1 | 1:2 | 1:4 | 1:8 |
| 1 | + | + | + | + |
| 2 | + | + | + | − |
| 3 | + | + | − | − |
| 4 | + | + | − | − |
| 5 | + | + | − | − |
| 6 | + | + | + | − |
| Positive control | + | + | + | + |
| Negative control 1 | − | − | − | − |
| Negative control 2 | − | − | − | − |
| Blank control | − | − | − | − |

The experimental results showed that Vero and MDCK cell lines stably expressing the four genes constructed by single- or double- plasmid can be used for virus rescue and can produce intact virus hemagglutination activity under undiluted or 2-fold dilution conditions. This result further proves that the virus rescue is successful. Among them, the use of single plasmid-integrated cell using Vero as a host cell has better efficiency as a host cell for virus rescue.

1. A method for rescuing an influenza virus, characterized in that a mammalian host cell stably expressing influenza virus PA, PB1, PB2 and NP genes is provided, and an influenza virus rescue system comprising mutant PA, PB1, PB2 and NP genes is introduced into the aforementioned host cell to achieve rescue, wherein the mutations make the influenza virus rescue system unable to rescue intact virus in natural mammalian cells.

2. The method according to claim 1, wherein the method comprises the following steps:
(1) constructing a single- or multiple-plasmid system encoding the PA, PB1, PB2 and NP genes;
(2) introducing the single- or multiple-plasmid system of step (1) into a mammalian cell, and screening a host cell stably expressing the four genes;
(3) constructing recombinant plasmids for the mutant PA, PB1, PB2 and NP genes and recombinant plasmids encoding the four genes HA, NA, M and NS respectively to form an influenza virus rescue system, the mutations are achieved by introducing a TAG codon into each of the four gene sequences;
(4) co-transfecting the influenza virus rescue system constructed in step (3) into the host cell of step (2);
(5) culturing the cell obtained in step (4) and harvesting the particles of the influenza virus.

3. The method according to claim 2, wherein the nucleotide sequences of the PA, PB1, PB2 and NP genes in step (1) are as shown in SEQ ID NO: 1, SEQ ID NO: 2, SEQ ID NO:3 and SEQ ID NO: 4, respectively.

4. An influenza virus prepared by the method according to claim 1.

5. A method for preventing or treating influenza, comprising administering the influenza virus according to claim 4.

6. The method according to claim 2, wherein in step (2), the single- or multiple-plasmid system of step (1) is introduced into a mammalian cell by electrotransformation.

7. The method according to claim 2, wherein the PA, PB1, PB2 and NP genes introduced into the host cell and the HA, NA, M and NS genes in the influenza virus rescue system are all derived from the A/WSN/1933 strain of influenza virus H1N1.

8. The method according to claim 2, wherein the influenza virus rescue system comprises the following eight plasmids: pPolI-M-PB2, pPolI-M-PB1, pPolI-M-PA, pPolI-M-NP, pPolI-WSN-HA, pPolI-WSN-NA, pPolI-WSN-M and pPolI-WSN-NS, wherein, the PA gene contained has a mutation at R266 codon to TAG, the PB1 gene contained has a mutation at R52 codon to TAG, the PB2 gene contained has a mutation at K33 codon to TAG, and the NP gene contained has a mutation at D101 codon to TAG.

9. The method according to claim 2, wherein the mammalian cell is selected from the group consisting of Vero cells, MDCK cells, 293 cells or MRCS cells.

10. The method according to claim 1, wherein, the PA, PB1, PB2 and NP genes are all derived from the A/WSN/1933 strain of influenza virus H1N1.

11. The method according to claim 1, wherein the nucleotide sequences of the PA, PB1, PB2 and NP genes are as shown in SEQ ID NO: 1, SEQ ID NO: 2, SEQ ID NO:3 and SEQ ID NO: 4, respectively.

12. The method according to claim 1, wherein the influenza virus rescue system comprises the following eight plasmids: pPolI-M-PB2, pPolI-M-PB1, pPolI-M-PA, pPolI-M-NP, pPolI-WSN-HA, pPolI-WSN-NA, pPolI-WSN-M and pPolI-WSN-NS, wherein, the PA gene contained has a mutation at R266 codon to TAG, the PB1 gene contained has a mutation at R52 codon to TAG, the PB2 gene contained has a mutation at K33 codon to TAG, and the NP gene contained has a mutation at D101 codon to TAG.

13. The method according to claim 1, wherein the mammalian host cell is selected from the group consisting of Vero cells, MDCK cells, 293 cells or MRCS cells.


SEQUENCE LISTING (22 sequences)

SEQ ID NO: 1 - DNA, 2151 bp, Artificial Sequence
  Codon-optimized PA gene of A/WSN/1933 of influenza virus H1N1
atggaggacttcgtgaggcagtgcttcaaccccatgatcgtggagctggccgagaaggccatgaaggagtacggcgaggacctgaagatcgagaccaacaagttcgccgccatctgcacacacctggaggtgtgcttcatgtactctgacttccacttcatcgacgagcagggcgagtccatcgtggtggagctgggggaccccaacgctctgctgaagcaccggttcgagatcatcgagggacgggacaggaccatcgcctggacagtgatcaacagcatctgcaacaccacaggggccgagaagcccaagttcctgcccgacctgtacgactacaagaagaacaggttcatcgagatcggcgtgaccaggagagaggtgcacatctactacctggagaaggccaacaagatcaagtccgagaagacccacatccacatcttcagcttcacaggggaggagatggccaccaaggccgactacacactggacgaggagagcagggcccggatcaagaccaggctgttcacaatcagacaggagatggcctccaggggcctgtgggactccttccggcagagcgagaggggagaggagacaatcgaggagagattcgagatcaccggcacaatgagaaagctggccgaccagtccctgccacccaacttcagctctctggagaagttcagagcctacgtggacgggttcgagcccaacggctacatcgaggggaagctgagccagatgtctaaggaggtgaacgccagaatcgagcccttcctgaagagcacccccaggcccctgagactgccagacggaccaccatgctcccagcggagcaagttcctgctgatggacgccctgaagctgtccatcgaggaccccagccacgagggagagggcatccccctgtacgacgccatcaagtgcatgagaacattcttcggctggaaggagcccaacgtggtgaagccccacgagaaggggatcaaccccaactacctgctgagctggaagcaggtgctggccgagctgcaggacatcgagaacgaggagaagatcccccggaccaagaacatgaagaagacatctcagctgaagtgggctctgggagagaacatggctccagagaaggtggacttcgacgactgcaaggacgtgggggacctgaagcagtacgacagcgacgagcccgagctgaggtctctggcctcctggattcagaacgagttcaacaaggcctgcgagctgaccgactccagctggatcgagctggacgagatcggagaggacgctgctcccatcgagcacatcgccagcatgcggaggaactacttcaccgccgaggtgtctcactgcagggccacagagtacatcatgaagggcgtgtacatcaacacagccctgctgaacgcctcctgcgctgctatggacgacttccagctgatccccatgatcagcaagtgcagaaccaaggaggggagacggaagacaaacctgtacggcttcatcatcaagggcaggagccacctgcggaacgacaccgacgtggtgaacttcgtgtctatggagttctccctgacagacccccggctggagccacacaagtgggagaagtactgcgtgctggaggtcggcgacatgctgctgaggtctgccatcgggcacgtgtcccggcccatgttcctgtacgtgaggaccaacggcacaagcaagatcaagatgaagtgggggatggagatgaggagatgcctgctgcagagcctgcagcagatcgagtctatgatcgaggccgagtcttccgtgaaggagaaggacatgaccaaggagttcttcgagaacaagtccgagacatggccagtgggagagagcccaaagggagtggaggagggctctatcgggaaggtgtgccggaccctgctggccaagagcgtgttcaactctctgtacgcctccccacagctggagggcttcagcgccgagtctaggaagctgctgctgatcgtgcaggccctgagagacaacctggagccagggaccttcgacctgggagggctgtacgaggccatcgaggagtgcctgatcaacgacccctgggtgctgctgaacgccagctggttcaactctttcctgacacacgccctgagatga

SEQ ID NO: 2 - DNA, 2274 bp, Artificial Sequence
  Codon-optimized PB1 gene of A/WSN/1933 of influenza virus H1N1
atggacgtgaaccccacactgctgttcctgaaggtgcccgcccagaacgccatctccaccacattcccctacaccggcgaccccccatacagccacggaaccgggacaggctacaccatggacacagtgaacaggacacaccagtactctgagagagggcggtggaccacaaacaccgagacaggagctccacagctgaaccccatcgacggaccactgccagaggacaacgagccatccggatacgctcagaccgactgcgtgctggaggccatggccttcctggaggagagccaccccgggatcttcgagacctcttgcctggagacaatggaggtggtgcagcagacccgggtggacaagctgacacagggcaggcagacctacgactggacactgaacagaaaccagccagctgctaccgccctggccaacacaatcgaggtgttcagatccaacggactgaccgctaacgagagcggccggctgatcgacttcctgaaggacgtgatggagtctatgaacaaggaggagatggagatcaccacacacttccagagaaagaggagagtgcgggacaacatgacaaagaagatggtgacccagagaacaatcggcaagagaaagcagcggctgaacaagaggagctacctgatcagagccctgaccctgaacaccatgacaaaggacgctgagagagggaagctgaagagaagggccatcgccaccccagggatgcagatcaggggcttcgtgtacttcgtggagacactggccaggagcatctgcgagaagctggagcagtccggactgccagtgggagggaacgagaagaaggccaagctggccaacgtggtgcggaagatgatgaccaactctcaggacacagagatcagcttcaccatcacaggggacaacacaaagtggaacgagaaccagaaccccaggatgttcctggccatgatcacctacatcacacggaaccagcccgagtggttcaggaacgtgctgtccatcgcccccatcatgttcagcaacaagatggccagactggggaagggctacatgttcgagtctaagtccatgaagatcagaacccagatcccagctgagatgctggccagcatcgacctgaagtacttcaacgactctaccagaaagaagatcgagaagatcagaccactgctgatcgacggaacagccagcctgtctcccgggatgatgatgggcatgttcaacatgctgagcaccgtgctgggggtgtctatcctgaacctgggccagaagaggcacacaaagaccacatactggtgggacggcctgcagagctctgacgacttcgccctgatcgtgaacgctccaaaccacgagggcatccaggctggcgtgaacaggttctacagaacatgcaagctgctggggatcaacatgagcaagaagaagagctacatcaaccggaccggcacattcgagttcacctctttcttctaccgatacgggttcgtggccaacttctccatggagctgccctctttcggggtgtccggcatcaacgagagcgccgacatgtctatcggcgtgaccgtgatcaagaacaacatgatcaacaacgacctgggaccagctacagctcagatggccctgcagctgttcatcaaggactacaggtacacctaccggtgccacaggggcgacacccagatccagacaagacggagcttcgagatcaagaagctgtgggagcagacccactctaaggctggactgctggtgtccgacggaggacccaacctgtacaacatcaggaacctgcacatccccgaggtgtgcctgaagtgggagctgatggacgaggactaccagggcagactgtgcaaccccctgaaccccttcgtgaaccacaaggacatcgagtccgtgaacaacgccgtgatcatgccagctcacggaccagctaagaacatggagtacgacgccgtggccaccacacacagctggattcccaagaggaacagatccatcctgaacaccagccagaggggcatcctggaggacgagcagatgtaccagaagtgctgcaacctgttcgagaagttcttcccctccagctcttacaggagacccgtgggcatctccagcatggtggaggctatggtgagccgggccaggatcgacgccaggatcgacttcgagtctgggagaatcaagaaggaggagttcaccgagatcatgaaaatctgcagcacaatcgaggagctgcggaggcagaagtga

SEQ ID NO: 3 - DNA, 2280 bp, Artificial Sequence
  Codon-optimized PB2 gene of A/WSN/1933 of influenza virus H1N1
atggagaggatcaaggagctgagaaacctgatgtctcagtcccggacaagggagatcctgaccaagaccacagtggaccacatggccatcatcaagaagtacaccagcggcaggcaggagaagaaccccgccctgagaatgaagtggatgatggccatgaagtaccccatcacagccgacaagcggatcaccgagatgatccccgagaggaatgagcagggccagaccctgtggagcaagatgaacgacgccggcagcgacagagtgatggtgagccccctggccgtgacatggtggaacagaaacggccccgtgacatctaccgtgcactaccccaaaatctacaagacatacttcgagaaggtggagagactgaagcacggcaccttcgggccagtgcacttccggaaccaggtgaagatcaggagacgggtggacatcaacccaggacacgccgacctgtctgccaaggaggcccaggacgtgatcatggaggtggtgttccccaacgaagtgggcgccagaatcctgaccagcgagtctcagctgaccacaaccaaggagaagaaggaggagctgcaggggtgcaagatcagccccctgatggtggcctacatgctggagcgggagctggtgagaaagacccggttcctgccagtggctggaggcaccagcagcgtgtacatcgaggtgctgcacctgacacagggcacctgctgggagcagatgtacacaccaggaggagaggctagaaacgacgacgtggaccagtccctgatcatcgccgccaggaacatcgtgaggagagctacagtgtctgccgaccccctggcctccctgctggagatgtgccacagcacccagatcggcggcatcaggatggtgaacatcctgaggcagaaccccacagaggagcaggccgtggacatctgcaaggccgccatgggcctgaggatctccagctctttctccttcggcgggttcacattcaagagaaccagcggctccagcgtgaagagagaggaggaggtgctgacagggaacctgcagaccctgaagatcagggtgcacgagggctacgaggagttcacaatggtggggcggagggctaccgctatcctgagaaaggccacaagacggctgatccagctgatcgtgagcggccgggacgagcagtctatcgccgaggccatcatcgtggccatggtgttcagccaggaggactgcatgatcaaggccgtgaggggggacctgaacttcgtgaaccgggccaaccagaggctgaaccccatgcaccagctgctgagacacttccagaaggacgccaaggtgctgttccagaactggggcatcgagtctatcgacaacgtgatgggcatgatcggcatcctgcccgacatgacaccctccaccgagatgagcatgaggggcgtgagaatctccaagatgggggtggacgagtactcttccgccgagaagatcgtggtgagcatcgacaggttcctgagagtgcgggaccagagaggcaacgtgctgctgtctcccgaggagatcagcgagacacaggggaccgagaagctgacaatcacctacagctcttccatgatgtgggagatcaacggccccgagtctgtgctggtgaacacctaccagtggatcatcagaaactgggagacagtgaagatccagtggtcccagaaccccaccatgctgtacaacaagatggagttcgagcccttccagagcctggtgccaaaggccgtgaggggccagtactctgggttcgtgagaaccctgttccagcagatgcgggacgtgctgggcacattcgacaccgcccagatcatcaagctgctgcccttcgccgccgccccaccaaagcagagccggacacagttcagctctctgaccatcaacgtgaggggctctgggatgagaatcctggtgcgggggaactcccccgtgttcaactacaacaagacaaccaagcggctgaccgtgctgggcaaggacgctggaccactgaccgaggaccccgacgagggcacagccggggtggagagcgccgtgctgaggggcttcctgatcctggggaaggaggacaggagatacggccccgccctgagcatcaacgagctgtctaacctggccaagggggagaaggccaacgtgctgatcggccagggcgacgtggtgctcgtgatgaagaggaagagaaactccagcatcctgaccgacagccagacagccaccaagcggatcaggatggccatcaactga

SEQ ID NO: 4 - DNA, 1497 bp, Artificial Sequence
  Codon-optimized NP gene of A/WSN/1933 of influenza virus H1N1
atggccaccaagggcacaaagaggagctacgagcagatggagaccgacggggagaggcagaacgccacagagatcagagcctctgtgggcaagatgatcgacggcatcgggagattctacatccagatgtgcaccgagctgaagctgtccgactacgaggggagactgatccagaactctctgacaatcgagcggatggtgctgtccgccttcgacgagaggagaaacaagtacctggaggagcacccaagcgccggcaaggaccccaagaagaccggaggaccaatctacagaagggtggacgggaagtggagacgggagctgatcctgtacgacaaggaggagatcaggagaatctggaggcaggctaacaacggcgacgacgctaccgctgggctgacacacatgatgatctggcactctaacctgaacgacgccacctaccagaggacaagagccctggtgcggaccggaatggaccccaggatgtgctctctgatgcaggggtccacactgccacggaggagcggagctgctggagctgccgtgaagggcgtggggaccatggtcatggagctgatccggatgatcaagaggggcatcaacgacagaaacttctggcggggcgagaacgggagacggacaaggatcgcctacgagagaatgtgcaacatcctgaagggcaagttccagaccgccgcccagaggacaatggtggaccaggtgagggagagcaggaaccccggcaacgccgagttcgaggacctgatcttcctggctagatccgccctgatcctgagagggagcgtggctcacaagtcttgcctgccagcttgcgtgtacggcagcgccgtggcctctgggtacgacttcgagagggagggctacagcctggtggggatcgaccccttcagactgctgcagaactcccaggtgtacagcctgatcagaccaaacgagaacccagctcacaagagccagctggtgtggatggcctgccactctgccgccttcgaggacctgagggtgagctctttcatcagaggaaccaaggtggtgccccggggcaagctgtccacaaggggggtgcagatcgccagcaacgagaacatggagaccatggagtccagcacactggagctgcggtccaggtactgggccatcagaacccggagcggcgggaacacaaaccagcagagggcctcttccggccagatcagcatccagcccaccttcagcgtgcagagaaacctgcccttcgaccggcccacaatcatggctgccttcaccggaaacacagagggacggaccagcgacatgaggacagagatcatcagactgatggagagcgccagaccagaggacgtgtctttccagggcaggggggtgttcgagctgtctgacgagaaggccacctcccccatcgtgccctctttcgacatgtccaacgagggcagctacttcttcggggacaacgccgaggagtacgacaactga

SEQ ID NO: 5 - DNA, 4595 bp, Artificial Sequence
  Plasmid pBudCE4.1
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcattcctgcaggtcgacatcgatcttaagcagtacttctagaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcttcgaaggtaccagcacagtggactcgagagatctggccggctgggcccgtttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 6 - DNA, 600 bp, Artificial Sequence
  Puro reporter gene
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctag

SEQ ID NO: 7 - DNA, 41 bp, Artificial Sequence
  Primer
ggacctatatacaggagagtatagggaaagtggaggagaga

SEQ ID NO: 8 - DNA, 41 bp, Artificial Sequence
  Primer
tctctcctccactttccctatactctcctgtatataggtcc

SEQ ID NO: 9 - DNA, 50 bp, Artificial Sequence
  Primer
gcccatccggaagtctaagtggctatggtgttgatttcaaaaaaggttca

SEQ ID NO: 10 - DNA, 50 bp, Artificial Sequence
  Primer
tgaaccttttttgaaatcaacaccatagccacttagacttccggatgggc

SEQ ID NO: 11 - DNA, 39 bp, Artificial Sequence
  Primer
ctgtcttcctgatgtgtactacttgattatggccatatg

SEQ ID NO: 12 - DNA, 39 bp, Artificial Sequence
  Primer
catatggccataatcaagtagtacacatcaggaagacag

SEQ ID NO: 13 - DNA, 42 bp, Artificial Sequence
  Primer
gtttgttgtccatcttccctattctgagtactgatgtgtcct

SEQ ID NO: 14 - DNA, 42 bp, Artificial Sequence
  Primer
aggacacatcagtactcagaatagggaagatggacaacaaac

SEQ ID NO: 15 - DNA, 18 bp, Artificial Sequence
  Primer
gatggagaacgccagaat

SEQ ID NO: 16 - DNA, 20 bp, Artificial Sequence
  Primer
gcatcgtcaccattattagc

SEQ ID NO: 17 - DNA, 20 bp, Artificial Sequence
  Primer
accaggctattcaccataag

SEQ ID NO: 18 - DNA, 21 bp, Artificial Sequence
  Primer
ccttccatccaaagaatgttc

SEQ ID NO: 19 - DNA, 19 bp, Artificial Sequence
  Primer
ggacaggaacaggatacac

SEQ ID NO: 20 - DNA, 18 bp, Artificial Sequence
  Primer
tgctgaacaacctccatc

SEQ ID NO: 21 - DNA, 23 bp, Artificial Sequence
  Primer
cgaaagcaggtcaattatattca

SEQ ID NO: 22 - DNA, 20 bp, Artificial Sequence
  Primer
tacttgtcactggtccattc